(19) United States
(12) Patent Application Publication
(10) Pub. No.: US 2022/0138945 A1
(43) Pub. Date: May 5, 2022

(54) Methods for Preparing Data from Tissue Sections for Machine Learning Using Both Brightfield and Fluorescent Imaging

(71) Applicant: Flagship Biosciences, Inc., Westminster, CO (US)

(72) Inventors: Cris L. Luengo Hendriks, Westminster, CO (US); Roberto Gianani, Lakewood, CO (US)

(73) Assignee: Flagship Biosciences, Inc., Westminster, CO (US)

(21) Appl. No.: 17/576,349

(22) Filed: Jan. 14, 2022

Related U.S. Application Data
(63) Continuation-in-part of application No. 16/271,525, filed on Feb. 8, 2019.
Application No. 16/271,525 is a continuation-in-part of application No. 15/396,552, filed on Dec. 31, 2016, now abandoned.

(51) Int. Cl. G06T 7/00; G16H 50/20; G16H 30/40; G06V 20/69; G06V 20/70; G06V 10/774; G01N 1/30

(52) U.S. Cl. CPC G06T 7/0012; G16H 50/20; G16H 30/40; G06V 20/695; G06V 20/70; G01N 2001/302; G01N 1/30; G06T 2207/10064; G06T 2207/30024; G06T 2207/20081; G06V 10/774

(57) ABSTRACT

In digital pathology, obtaining a labeled data set for training, testing and/or validation of a machine learning model is expensive, because it requires manual annotations from a pathologist. In some cases, it can be difficult for the pathologist to produce correct annotations. The present invention allows the creation of labeled data sets using fluorescent dyes, which do not affect the appearance of the slide in the brightfield imaging modality. It thus becomes possible to add correct annotations to a brightfield slide without human intervention.

### CROSS-REFERENCE TO RELATED APPLICATIONS

This application is a continuation-in-part (CIP) of U.S. Ser. No. 16/271,525, filed Sep. 30, 2020, and titled “Methods for Identification of Tissue Objects in IHC Without Specific Staining”, which is a CIP of U.S. Ser. No. 15/396,552, filed Dec. 31, 2016, and titled “METHODS FOR DETECTING AND QUANTIFYING MULTIPLE STAINS ON TISSUE SECTIONS”;

the contents of each of which are hereby incorporated by reference.

### BACKGROUND

### Field of the Invention

This invention relates generally to image analysis methods for the assessment of stained tissue sections. More specifically, the present invention relates to methods of preparing data from tissue sections for machine learning using both brightfield and fluorescent imaging.

### Description of the Related Art

Tissue sections are commonly made visible in a brightfield microscope using chromogenic dyes. One technique uses immunochemistry to localize the dye to where a specific biomarker is present (e.g., a protein or RNA molecule). Tissue sections can also be examined under the fluorescence microscope, after staining it with fluorescent dyes. Such dyes can also be localized to specific biomarkers using a similar technique referred to as immunofluorescence.

Methods are known to extract the contribution of individual chromogenic dyes from the color image obtained by a brightfield microscope. These methods only work if there are no more different dyes on the slide than color channels acquired by the microscope's camera. Since brightfield microscopes typically have RGB cameras, chromogenic dye quantification is typically limited to three dyes (two biomarkers and one counterstain for the nuclei). It is possible to use multispectral imaging to circumvent this limit, but it is a slow technique requiring specialized equipment.

In contrast, fluorescence microscopy is limited by the availability of fluorescent dyes with emission spectra that do not overlap. Each dye is typically imaged consecutively and independently. For the special case of one red, one green and one blue dye, one can use a special filter cube and an RGB camera to image all three dyes simultaneously. It is also possible to measure the emission spectra of the various dyes, and use that information to remove the channel cross-talk (caused by overlapping emission spectra), thereby increasing the amount of dyes that can be used together on the same tissue section.

Brightfield and fluorescence microscopes have a lot in common, and fluorescence microscopes usually have a brightfield mode. The two imaging modalities require different forms of illumination, and the fluorescence modality adds some filters to the light path. Sometimes a different camera will also be selected, though this is not necessary. Although whole slide scanners that can scan a slide in both brightfield and fluorescence modalities exist, technical differences between the modalities may not allow optimal approaches for interpreting staining from both modalities simultaneously.

Machine learning comprises a group of methods and algorithms to teach a computer to distinguish things. In the case of tissue sections, one could use these methods to teach a computer to distinguish different cell types, or to determine if the tissue sample is of healthy or cancerous tissue. Machine learning can be either very simple methods such as a linear classifier or a decision tree, or more complex ones such as random forests, support vector machines, or neural networks, including convolutional neural networks. Deep learning is a term commonly used today that refers to deep neural networks (networks with many hidden layers), and consequently is a form of machine learning.

### SUMMARY

In accordance with the embodiments herein, methods are described for preparing data from tissue sections for use with machine learning using both brightfield and fluorescent imaging. Generally, the method entails the following eight steps: (i) staining a tissue section with brightfield stain ensuring that a particular tissue object is stained; (ii) staining the same tissue section with fluorescent stain ensuring that the same tissue object is stained and that target cells are identified with the fluorescent stain; (iii) scanning the tissue section in brightfield and fluorescence to create two images; (iv) quantifying and identifying cells within the brightfield image; (v) creating a data set using a subset of the identified cells; (vi) aligning the fluorescent image with the brightfield image using the tissue object that is stained in both brightfield and fluorescent; (vii) labeling the cells in the data set based on the staining of the target cells in fluorescent; (viii) using the labeled cells within the data set for machine learning, for example to train a model to identify the target cells without specific staining. The same process can be followed without identifying individual cells, where the method identifies target regions of tissue.

### BRIEF DESCRIPTION OF THE DRAWINGS

FIG. 1 illustrates the general method for preparing data from tissue sections for machine learning using both brightfield and fluorescent imaging.

FIG. 2 illustrates a second method for preparing data from tissue sections for machine learning both brightfield and fluorescent imaging.

### DETAILED DESCRIPTION OF EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention.

For purpose of definition, a tissue object is one or more of a cell (e.g., immune cell), cell sub-compartment (e.g., nucleus, cytoplasm, membrane, organelle), cell neighborhood, a tissue compartment (e.g., tumor, tumor microenvironment (TME), stroma, lymphoid follicle, healthy tissue), blood vessel, a lymphatic vessel, vacuole, collagen, regions of necrosis, extra-cellular matrix, a medical device (e.g., stent, implant), a gel, a parasitic body (e.g., virus, bacterium,), a nanoparticle, a polymer, and/or a non-dyed object (e.g., metal particle, carbon particle). Tissue objects are visualized by histologic stains which highlight the presence and localization of a tissue object. Tissue objects can be identified directly by stains specifically applied to highlight the presence of said tissue object (e.g., hematoxylin to visualize nuclei, immunohistochemistry stain for a protein specifically found in a muscle fiber membrane), indirectly by stains applied which non-specifically highlight the tissue compartment (e.g., DAB background staining), are biomarkers known to be localized to a specific tissue compartment (e.g., nuclear-expressed protein, carbohydrates only found in the cell membrane), or can be visualized without staining (e.g., carbon residue in lung tissue).

For the purpose of this disclosure, patient status includes diagnosis of inflammatory status, disease state, disease severity, disease progression, therapy efficacy, and changes in patient status over time. Other patient statuses are contemplated.

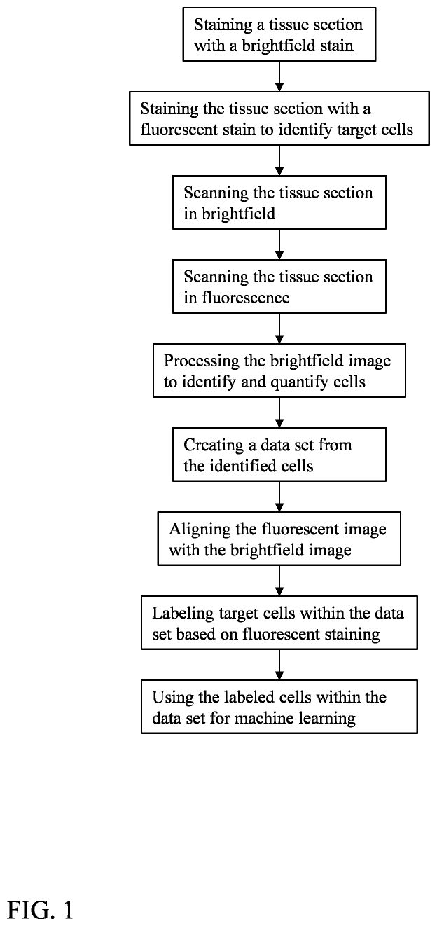

In an illustrative embodiment of the invention, the methods can be summarized in the following eight steps: (i) staining a tissue section with brightfield stain ensuring that a particular tissue object is stained; (ii) staining the same tissue section with fluorescent stain ensuring that the same tissue object is stained and that target cells are identified with the fluorescent stain; (iii) scanning the tissue section in brightfield and fluorescence to create two images; (iv) quantifying and identifying cells within the brightfield image; (v) creating a data set using a subset of the identified cells; (vi) aligning the fluorescent image with the brightfield image using the tissue object that is stained in both brightfield and fluorescent; (vii) labeling the cells in the data set based on the staining of the target cells in fluorescent; (viii) using the labeled cells within the data set for machine learning. This illustrative embodiment of the invention is summarized in FIG. 1. The invention thus results in a data set that can be used to train (or test, or validate) a machine learning model to identify cells of interest in a brightfield image, without adding a specific stain to that brightfield image. The model would then be applied to images of slides that have not had the fluorescence stains added.

In some embodiments the subset of identified cells is all of the identified cells. In other embodiments, the machine learning is training a machine learning model to identify the target cells, testing the machine learning model, or validating the trained machine learning model.

In further embodiments, the machine learning model is used to identify a patient status for a patient from whom the tissue section was taken or for a separate patient not associated with the tissue section used to train the machine learning model. This embodiment can be used to create a “synthetic stain”, a markup of a digital image of a tissue section that has not been stained to cause the cells within that digital image to appear as if they had been stained.

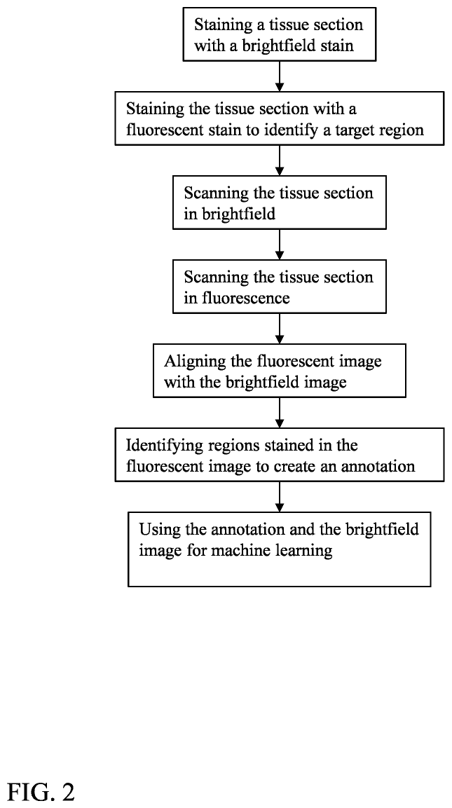

Another embodiment of the invention is illustrated in FIG. 2 and is summarized in the following five steps: (i) staining a tissue section with a brightfield stain; (ii) staining the same tissue section with a fluorescent stain that identifies target tissue regions; (iii) scanning the tissue section in both brightfield and fluorescence to create two images; (iv) aligning the fluorescent image to the brightfield image; (v) identifying regions stained in the fluorescent image to create an annotation; and (vi) using the annotation and the first image for machine learning. In this embodiment, the resulting data set annotates specific tissue regions in the brightfield image.

In further embodiments, the machine learning is training a machine learning model to identify the target tissue region, testing the machine learning model, or validating the trained machine learning model.

In further embodiments, the machine learning model is used to identify a patient status for a patient from whom the tissue section was taken or for a separate patient not associated with the tissue section used to train the machine learning model. This embodiment can be used to create a “synthetic stain”, a markup of a digital image of a tissue section that has not been stained to cause the cells within that digital image to appear as if they had been stained.

Any of these embodiments can be used with multiple tissue sections to feed into the data set. This improves the accuracy and precision of the machine learning.

What is claimed is:

1. A method comprising:
staining a tissue section with at least one brightfield stain, wherein the at least one brightfield stain includes staining for at least one tissue object;
staining the tissue section with at least one fluorescent stain, wherein the at least one fluorescent stain includes staining for the at least one tissue object and identifies target cells;
scanning the tissue section in brightfield to create a first image;
scanning the tissue section in fluorescence to create a second image;
processing the first image to identify and quantify cells within the tissue section;
creating a data set of a subset of the identified cell within the tissue section;
aligning the second image to the first image using the at least one tissue object;
labeling the cells within the data set based on staining of the target cells; and
using the labeled cells within the data set for machine learning.

2. The method of claim 1, wherein the subset of the identified cells is all identified cells.

3. The method of claim 1, wherein the machine learning is training a machine learning model to identify the target cells.

4. The method of claim 3, wherein the machine learning is testing the machine learning model.

5. The method of claim 4, wherein the machine learning is validating the machine learning model.

6. The method of claim 5, further comprising using the machine learning model to identify a patient status for a patient selected form the group consisting of from whom the tissue section was taken and unrelated to the tissue section used for training the machine learning model.

7. The method of claim 6, wherein the patient status for a patient unrelated to the tissue section used for training the machine learning model is determined via the use a synthetic stain applied to a digital image of an unstained tissue section taken from that patient.

8. The method of claim 1, further comprising applying the machine learning to a digital image of an unstained tissue section to create a synthetic stain on the digital image to identify target cells within that digital image.

9. A method comprising:
staining a tissue section with at least one brightfield stain;
staining the tissue section with at least one fluorescent stain, wherein the at least one fluorescent stain identifies at least one target tissue region;
scanning the tissue section in brightfield to create a first image;
scanning the tissue section in fluorescence to create a second image;
aligning the second image to the first image;
identifying regions stained in the second image to create an annotation; and
using the annotation and the first image for machine learning.

10. The method of claim 9, wherein the machine learning is training a machine learning model to identify the at least one target tissue region.

11. The method of claim 10, wherein the machine learning is testing the machine learning model.

12. The method of claim 11, wherein the machine learning is validating the machine learning model.

13. The method of claim 12, further comprising using the machine learning to identify a patient status for a patient selected form the group consisting of from whom the tissue section was taken and unrelated to the tissue section used for training the machine learning model.

14. The method of claim 13, wherein the patient status for a patient unrelated to the tissue section used for training the machine learning model is determined via the use a synthetic stain applied to a digital image of an unstained tissue section taken from that patient.

15. The method of claim 9, further comprising applying the machine learning to a digital image of an unstained tissue section to create a synthetic stain on the digital image to identify target cells within that digital image.
